(19) United States
(12) Patent Application Publication
(10) Pub. No.: US 2023/0285067 A1
(43) Pub. Date: Sep. 14, 2023

(54) SURGICAL INSTRUMENT

(71) Applicant: GYRUS MEDICAL LIMITED, Cardiff (GB)

(72) Inventors: Nicholas TULP, Cardiff (GB); Nathan Philip Blake, Cardiff (GB)

(73) Assignee: GYRUS MEDICAL LIMITED, Cardiff (GB)

(21) Appl. No.: 18/183,284

(22) Filed: Mar. 14, 2023

Related U.S. Application Data
(60) Provisional application No. 63/319,498, filed on Mar. 14, 2022.

(51) Int. Cl. A61B 18/14

(52) U.S. Cl. CPC A61B 18/14; A61B 2018/00601

(57) ABSTRACT

A surgical instrument, comprising a motor drive unit comprising a motor drive unit; a rotary surgical end effector arranged to be coupled to the motor drive unit; and a coupling means for coupling the motor drive unit and the rotary surgical end effector, the coupling means being configured such that there is only one possible rotational alignment between the rotary surgical end effector and the motor drive unit when coupled. This arrangement guarantees the rotational position of the rotary surgical end effector in relation to the rotational position of the motor drive unit. This enables quick and easy control of the rotational position of the rotary surgical end effector.

### TECHNICAL FIELD

Embodiments of the present invention described herein relate to a surgical instrument, and more particularly to an arrangement for connecting a motor drive unit to a rotary shaver arrangement of the surgical instrument.

### BACKGROUND TO THE INVENTION AND PRIOR ART

Surgical apparatus are powered to enhance shaving, cutting and/or removal of tissue, bone and/or other bodily material. Such powered surgical apparatus can include a shaving or cutting instrument, such as a rotating blade, for example. The rotating blade can be connected to a handpiece which is held by an operator of the apparatus, such as a surgeon, for example. The surgeon, by holding the handpiece in the surgeon's hand, can thereby manipulate the rotating blade to shave or cut desired tissue, bone and/or other bodily material.

Surgical apparatus used to shave, cut, resect, abrade and/or remove tissue, bone and/or other bodily materials are known. Such surgical apparatus can include a cutting surface, such as a rotating blade disposed on an elongated inner tube that is rotated within an elongated outer tube having a cutting window. The inner and outer tubes form a surgical cutting instrument or unit. In general, the elongated outer tube includes a distal end defining an opening or cutting window disposed at a side of the distal end of the outer tube. The cutting window of the outer tube exposes the cutting surface of the inner tube (located at a side of the distal end of the inner tube) to tissue, bone and/or any other bodily materials to be removed. A powered handpiece is used to rotate the inner tube with respect to the outer tube while an outer tube hub (connected to the proximal end of the outer tube) is fixed to the handpiece and an inner tube hub (connected to the proximal end of the inner tube) is loosely held in place by the powered handpiece.

In some instruments the inner tube is hollow and has a cutting window on a side surface of its distal end such that tissue, bone, etc. will be cut or shaved as the cutting window of the inner tube aligns with and then becomes misaligned with the cutting window of the outer tube as the inner tube is rotated within the outer tube. The cutting device removes small pieces of the bone, tissue, etc. as the inner tube is rotated within the outer tube.

In some instruments a vacuum is applied through the inner tube such that the bodily material that is to be cut, shaved, etc. is drawn into the windows of the inner and outer tubes when those windows become aligned, thereby facilitating the cutting, shaving, etc. of the tissue, which then travels through the inner tube due to the suction.

Many times during surgery, the surgeon wishes to apply suction to the surgical site without performing cutting with the surgical instrument. This usually is done by withdrawing the surgical instrument and inserting a dedicated suction device (for example, a suction wand which is a tube to which suction is applied). However, exchanging the surgical tool for the dedicated suction device is time-consuming. Furthermore, insertion and removal of instruments into the patient can cause trauma and irritation to the passage of the patient, and thus it is desirable to minimize the number of times that surgical instruments need to be withdrawn and inserted/reinserted into the patient. Some instruments combine a shaver device and a radio frequency (RF) suction device to form an RF shaver device. Such a device has mechanical shaver functionality and RF functionality. Owing to the nature of an RF shaver, notably the suction requirements during RF only use, the control of the blade (inner tubular member) position is required to ensure a suction path is possible when the shaver blade is not spinning. Prior art arrangements require either (1) the blade to be spun until the rotor is in the necessary position, an action which can take several motor pulses to achieve; or (2) the blade is locked into either a fully open position or a fully closed position, this requires a visible check to ensure it is in the right state before operating.

FIGS. 1 and 2 show prior art arrangements which use a plastic hub to attach the instrument to the handpiece. The plastic hubs have 180-degree rotational symmetry, allowing the inner rotating blade to be in one of two positions relative to the handpiece shaft.

### SUMMARY OF THE DISCLOSURE

The present disclosure addresses the above problem, by providing a surgical instrument which has been designed to guarantee the rotational or angular position of a rotary surgical end effector in relation to the rotational or angular position of a motor drive unit.

Embodiments of the present invention provide a coupling means for coupling a motor drive unit and a rotary surgical end effector, the coupling means being configured such that there is only one possible rotational alignment between the rotary surgical end effector and the motor drive unit when coupled.

In view of the above, from a first aspect, the present disclosure relates to a surgical instrument, comprising a motor drive unit; a rotary surgical end effector arranged to be coupled to the motor drive unit; and a coupling means for coupling the motor drive unit and the rotary surgical end effector, the coupling means being configured such that there is only one possible rotational alignment between the rotary surgical end effector and the motor drive unit when coupled.

Several advantages are obtained from embodiments according to the above-described aspect. For example, the guaranteed rotational alignment between the motor drive unit and the rotary surgical end effector means that there is a direct and fixed correspondence between the rotational position of the motor drive unit and the rotational position of the rotary surgical end effector. E.g., position 1 on the motor drive unit corresponds to position 1 on the rotary surgical end effector. Without the guaranteed rotational alignment, the motor drive unit and the rotary surgical end effector could be connected in any rotational alignment and therefore the rotational position of the motor drive unit would not always correspond to the rotational position of the rotary surgical end effector. E.g., position 1 on the motor drive unit may correspond to position 1 on the rotary surgical end effector when connected in one rotational alignment, but may correspond to position 2 on the rotary surgical end effector when connected in a different rotational alignment. The rotational alignments would vary based on how the motor drive unit and the rotary surgical end effector were coupled. It is advantageous for the rotational alignment of the motor drive unit and the rotary surgical end effector to be guaranteed, as if it is always known that motor position 1=end effector position 1, then putting the motor into position 1 will always mean that the end effector is in position 1, so the user does not need to visibly check to ensure the end effector is in the right state before operating.

In some embodiments, the coupling means is configured such that the rotary surgical end effector is rotationally locked with the motor drive unit when coupled, such that there is a one-to-one relationship between rotation of the motor drive unit and rotation of the rotary surgical end effector. The rotational locking combined with the only one possible rotational alignment means that, once coupled, the rotational position of the rotary surgical end effector is always known as long as the rotational position of the motor drive unit is known.

In some embodiments, the coupling means comprises a first engaging portion located at a distal end of the motor drive unit and a second engaging portion located at a proximal end of the rotary surgical end effector, the first and second engaging portions arranged to engage when coupled. This embodiment is advantageous as the engaging portions are configured such that there is only one possible rotational alignment between the rotary surgical end effector and the motor drive unit when the portions are engaged.

In some embodiments, the motor drive unit comprises a drive shaft which protrudes from the first engaging portion, and the rotary surgical end effector comprises a mating portion configured to receive the drive shaft, the mating portion being located within the second engaging portion, the drive shaft and the mating portion being configured to mate when the rotary surgical end effector and the motor drive unit are coupled. This embodiment is advantageous as the drive shaft transfers torque from the motor drive unit to the rotary surgical end effector when the drive shaft and the mating portion are mated. This enables the function of the rotary surgical end effector.

In some embodiments, the first and second engaging portions are complementarily shaped such that there is only one position around the rotational axis where the rotary surgical end effector and the motor drive unit are fully coupled. This is advantageous as this ensures there is only one possible rotational alignment between the rotary surgical end effector and the motor drive unit when coupled.

In some embodiments, the rotary surgical end effector and the motor drive unit being fully coupled requires the drive shaft and the mating portion to mate. This is advantageous as the mating of the drive shaft and the mating portion enables torque to be transferred between the motor and the end effector.

In some embodiments, the first and second engaging portions form a spiral arrangement. This is advantageous as the spiral arrangement ensures there is only one position around the rotational axis where the rotary surgical end effector and the motor drive unit are fully coupled, and thus ensures there is only one possible rotational alignment between the rotary surgical end effector and the motor drive unit when coupled. The spiral arrangement is such that each of the first and second engaging portions are asymmetrical but complimentary shapes. A bidirectional spiral promotes the interconnection to rotate to the low part of the adjoining spiral, thus guaranteeing the rotational alignment.

In some embodiments, the motor drive unit is encoded such that the rotary surgical end effector is positionable in one or more discrete rotational positions. The encoding on the motor drive unit is advantageous as this allows the rotary surgical end effector to be easily rotated to certain rotational positions which may be required for certain functions of the instrument. For example, the motor drive unit may be encoded to have two discrete rotational positions, position 1 and position 2. By virtue of the guaranteed rotational alignment of embodiments of the present invention, these discrete rotational positions directly correspond to discrete rotational positions of the rotary surgical end effector, position 1 and position 2 of the end effector. In the example of the dual sided RF shaver, the rotary surgical end effector (the inner tubular member) needs to be parked in a “closed” position (i.e. the cutting windows need to be anti-aligned) for the RF-only functionality to work. By having set rotational positions encoded on the motor drive unit, the rotary surgical end effector can easily be positioned in a required position due to the guaranteed rotational alignment between the motor drive unit and the rotary surgical end effector via the coupling means.

In some embodiments, the rotary surgical end effector comprises a rotary shaver arrangement. In some embodiments, the rotary shaver arrangement comprises an inner tubular member arranged to mechanically cut tissue when rotated. In some embodiments, the rotary shaver arrangement comprises an outer tubular member which concentrically surrounds the inner tubular member, the outer tubular member having a central passageway and a first cutting window at the distal end thereof, the inner tubular member being rotatably mounted in the central passageway of the outer tubular member, and the inner tubular member having a second cutting window at the distal end thereof. These embodiments describing the rotary shaver arrangement are advantageous as the rotating blade (inner tubular member) can be rotated to shave or cut desired tissue, bone and/or other bodily material. Tissue, bone, etc. will be cut or shaved as the (second) cutting window of the inner tube aligns with and then becomes misaligned with the (first) cutting window of the outer tube as the inner tube is rotated within the outer tube. In use, the rotary shaver arrangement removes small pieces of the bone, tissue, etc. as the inner tube is rotated within the outer tube.

In some embodiments, the first and second cutting windows align when the inner tubular member of the motor drive unit is positioned in a first position of the one or more discrete rotational positions. This is advantageous as it can be necessary to position the inner tubular member in specific rotational positions for particular functionalities of the device. When the cutting windows align, the suction path through from the cutting windows, through the central lumen of the inner tubular member to the suction pump. This can suction cut tissue away from the surgical site after it has been cut by the rotary shaver arrangement.

In some embodiments, the first and second cutting windows anti-align when the inner tubular member of the motor drive unit is positioned in a second position of the one or more discrete rotational positions. This is advantageous as it can be necessary to position the inner tubular member in specific rotational positions for particular functionalities of the device. For example, in the case of dual-sided RF shaver devices, for RF-only functionality, the inner tubular member needs to be positioned such that the cutting windows anti-align (i.e., close). This is because the RF-only functionality needs a suction path from the suction aperture on the RF side of the device, through the central lumen of the inner tubular member to the suction pump. If the cutting windows are aligned (i.e., open), then there is no suction path between the suction aperture and the suction pump as the suction will preferably act through the cutting windows.

In some embodiments, a central passageway of the inner tubular member provides a central suction lumen. This is advantageous as this allows cut/shaved tissue etc. to be suctioned away from the surgical site.

In some embodiments, the rotary shaver arrangement is such that, when in use, rotation of the inner tubular member within the outer tubular member causes a tissue cutting action of a cutting blade located on the first cutting window interacting with the second cutting window. This is advantageous as tissue, bone, etc. will be cut or shaved as the cutting window of the inner tube aligns with and then becomes misaligned with the cutting window of the outer tube as the inner tube is rotated within the outer tube. The arrangement removes small pieces of the bone, tissue, etc. as the inner tube is rotated within the outer tube.

In some embodiments, the surgical instrument further comprises a handpiece arranged to house the motor drive unit and wherein the handpiece is coupleable to the rotary surgical end effector via the coupling means. This is advantageous as the end effector can be connected to the handpiece which is held by an operator of the apparatus, such as a surgeon, for example. The surgeon, by holding the handpiece in their hand, can thereby manipulate the rotating blade to shave or cut desired tissue, bone and/or other bodily material.

In some embodiments, the coupling means further comprises a torque transfer means to increase the torque transfer capacity between the motor drive unit and the rotary surgical end effector. This is advantageous as torque is better transferred when torque transfer means are present, opposed to when they are not present.

In some embodiments, the torque transfer means comprises a protrusion extending from one of the first and second engaging portions, arranged to be received by the other of the first and second engaging portions. The torque transfer means has the advantage that the engaging portions are more firmly engaged, and therefore far less likely to come uncoupled.

In some embodiments, the protrusion comprises a pin and/or a lug. This is advantageous as the pin/lug can extend from one engaging portion to be received by the other. This provides a more secure coupling than the two engaging portions just being interlocked.

From a second aspect, the present disclosure relates to an electrosurgical instrument, comprising the surgical instrument according to any of the above embodiments relating to the first aspect, further comprising a radio frequency (RF) arrangement including an active electrode; and an operative shaft having RF electrical connections operably connected to the active electrode.

From a third aspect, the present disclosure relates to an electrosurgical system, comprising an RF electrosurgical generator; and an electrosurgical instrument according to the second aspect above, the arrangement being such that in use the RF electrosurgical generator supplies an RF coagulation or ablation signal via the RF electrical connections to the active electrode.

### BRIEF DESCRIPTION OF THE DRAWINGS

Embodiments of the invention will now be further described by way of example only and with reference to the accompanying drawings, wherein:

FIG. 1 illustrates a prior art Mitek bidirectional hub;

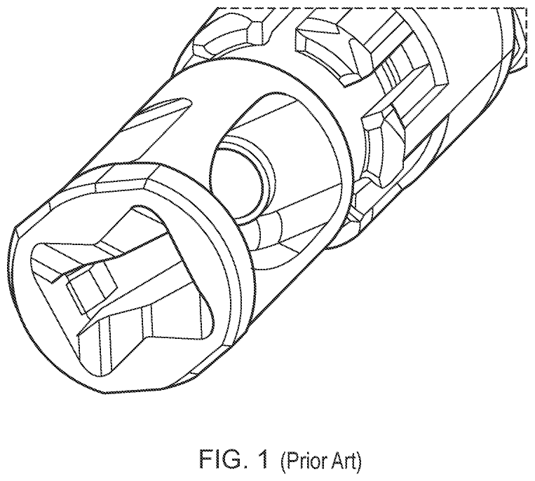

FIG. 2 illustrates a prior art Smith & Nephew bidirectional hub;

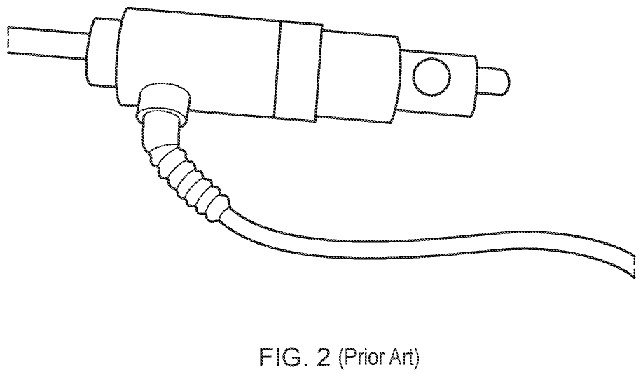

FIG. 3 is a schematic diagram of an electrosurgical system including an electrosurgical instrument;

FIG. 4 comprises sub-FIGS. 4A, 4B, and 4C illustrating respective engagement steps of embodiments of the present invention;

FIG. 5 illustrates a hub in accordance with some embodiments of the present invention with torque transfer features;

FIG. 6 is an end view of an electrosurgical instrument, in particular, an RF shaver device;

FIG. 7 is a perspective view of the RF shaver device;

FIG. 8 is a perspective view of the RF shaver device where the blade window is closed during use of the RF functionality;

FIG. 9 is a side view of the RF shaver device; and

FIG. 10 is a top view of the RF shaver device.

### DESCRIPTION OF THE EMBODIMENTS

Overview

Embodiments of the present invention provide a surgical instrument which has a connection configuration which guarantees the angular or rotational position of the rotary surgical end effector (e.g. a rotating shaver blade) with respect to the motor drive unit.

In more detail, the configuration guarantees the rotational position by only allowing one possible rotational alignment of the rotary surgical end effector and the motor drive unit.

The motor drive unit may be encoded such that the drive shaft of the motor drive unit is positionable in one or more discrete rotational positions. This means that, due to the fixed rotational alignment between the drive shaft and the rotary surgical end effector, the rotary surgical end effector is therefore also positionable in one or more discrete rotational positions, as it is rotationally fixed to the drive shaft when the shaft and the rotary surgical end effector are coupled to one another. Therefore, if the drive shaft is in a known discrete position, as encoded, the rotary surgical end effector will also be in a known discrete position.

This overcomes the problems of the prior art arrangements where the rotary surgical end effector's rotational or angular position is not discrete and known and/or fixed with respect to the drive shaft.

In the example where the rotary surgical end effector is an inner tubular member of a rotary shaver arrangement, the motor can be encoded such that a first position of the inner tubular member's one or more discrete rotational positions is such that the cutting window of the instrument is fully open. The motor can be encoded such that a second position of the inner tubular member's one or more discrete rotational positions is such that the cutting window of the instrument is fully closed. This is advantageous as, in the case of an RF shaver device, the RF-only functionality often requires that the cutting window of the instrument be fully closed for suction use to ensure a suction path extends to a suction aperture located on the RF side of the instrument, opposed to extending out of the cutting windows as with shaver functionality. Being able to put the inner tubular member into one or more discrete rotational positions avoids prior art issues of having to spin the inner tubular member until it is in the necessary position, an action which can take several motor pulses to get right, or having to visually check the positioning of the inner tubular member.

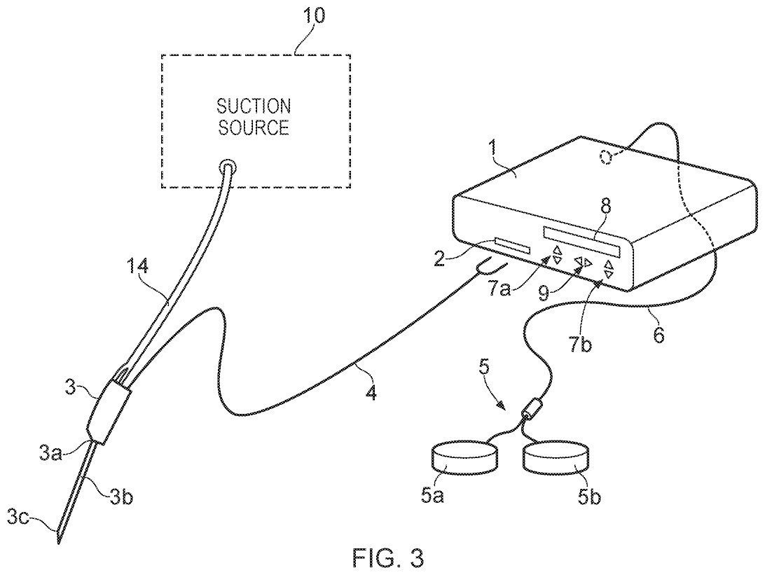

Various aspects and details of these principal components will be described below with reference to FIGS. 3 to 10. In the description below, the invention is described in relation to an electrosurgical instrument, in particular, an RF shaver device. However, the invention is equally applicable to any surgical instrument with a rotary surgical end effector. RF capabilities and/or shaver capabilities are not essential for the invention. The invention is about the connection between the motor and a rotary end effector.

The Electrosurgical System

Referring to the drawings, FIG. 3 shows an electrosurgical system including an electrosurgical generator 1 having an output socket 2 providing an RF output, via a connection cord 4, for an electrosurgical instrument 3. The instrument 3 has suction tubes 14 which are connected to a suction pump 10. Activation of the generator 1 may be performed from the instrument 3 via a handswitch (not shown) on the instrument 3, or by means of a footswitch unit 5 connected separately to the rear of the generator 1 by a footswitch connection cord 6. In the illustrated embodiment, the footswitch unit 5 has two footswitches 5a and 5b for selecting a coagulation mode or a cutting or vaporisation (ablation) mode of the generator 1 respectively. The generator front panel has push buttons 7a and 7b for respectively setting ablation (cutting) or coagulation power levels, which are indicated in a display 8. Push buttons 9 are provided as an alternative means for selection between the ablation (cutting) and coagulation modes.

The Electrosurgical Instrument

The instrument 3 includes a proximal hub portion 3a, a shaft 3b extending in a distal direction away from the proximal hub portion, and a distal end effector assembly 3c at the distal end of the shaft 3b. A power connection cord 4 connects the instrument to the RF generator 1. The instrument may further be provided with activation buttons (not shown), to allow the surgeon operator to activate either the mechanical cutting function of the end effector, or the electrosurgical RF functions of the end effector, which typically comprise coagulation or ablation.

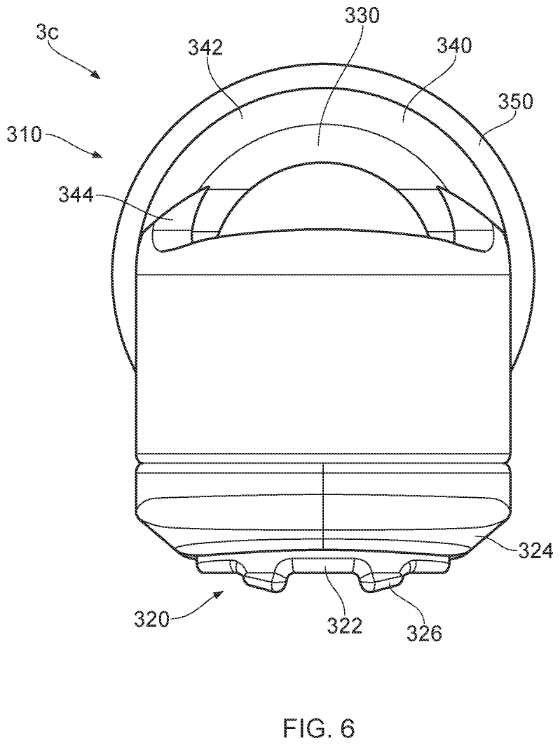

FIGS. 6 to 10 show an example of the distal end effector assembly 3c in more detail. The example electrosurgical instrument shown in FIGS. 6 to 10 is an RF shaver device which has both RF functionality and mechanical shaver functionality. The distal end effector 3c has two sides to it, the shaver side 310 and the RF side 320. The shaver side 310 is on the opposite side of the end effector to the RF side 320. The shaft 3b comprises an inner tubular member 330 and an outer tubular member 340. The outer tubular member 340 is concentrically surrounded by an insulative tubular member 350.

FIG. 6 shows an end view of the distal end effector assembly 3c. In FIG. 6, the blade window is closed. The inner tubular member 330 is co-axially disposed within an outer tubular member 340. The outer tubular member 340 has a larger diameter than the inner tubular member 330. The inner tubular member 330 has a proximal end and a distal end, with cutting window 332 (not visible in FIG. 6) disposed at a side of its distal end. The outer tubular member 340 also has a proximal end and a distal end, with cutting window 342 disposed at a side of its distal end. The edges of the cutting window 342 of the outer tubular member 340 comprise at least one sharpened edge to form a cutting blade. The at least one sharpened edge may be serrated such that it comprises teeth 344, as shown in the example given in the Figures.

The inner tubular member 330 is rotatably disposed inside of the outer tubular member 340 such that the surgical instrument 3 cuts tissue by rotating the inner tubular member 330 within the outer tubular member 340 while a vacuum is applied through the lumen of the inner tubular member 330 to draw the tissue into the cutting windows and sever the tissue by rotation of the inner tubular member 330.

The RF side 320 of the electrosurgical instrument 3 comprises an electrode assembly comprising an active electrode for tissue treatment (“active tip”) 322 received in a ceramic insulator 324. The active tip 322 is provided with projections 326 to concentrate the electric field at those locations. The projections 326 also serve to create a small separation between the planar surface of the active electrode 322 and the tissue to be treated. This allows conductive fluid to circulate over the planar surface, and avoids overheating of the electrode or the tissue. The active tip 322 of the instrument may be provided with a suction aperture (not shown), which is the opening to a lumen within an inner tubular member 330.

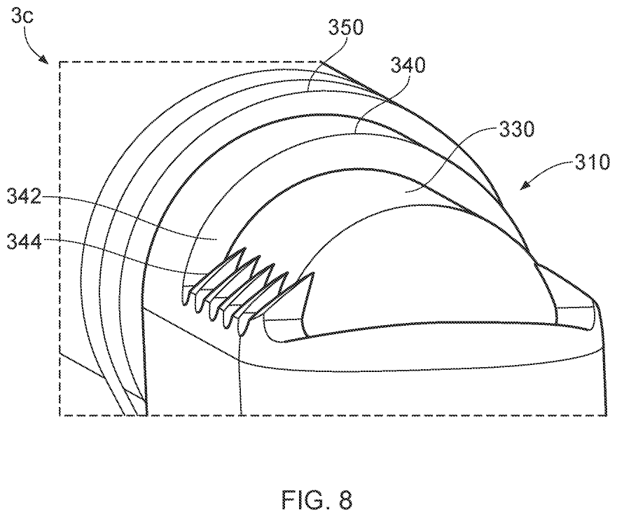

In more detail, when the RF side 320 is to be used as a suction tool by applying a vacuum through the lumen within the inner tubular member 330, the inner tubular member 330 (which acts as a cutting blade) is stopped from rotating and the cutting windows of the inner and outer tubular members are anti-aligned with each other, i.e. closing the cutting windows, (as is the case in FIG. 8) so that the vacuum is applied through the suction path connecting the suction aperture to the suction pump 10 via the lumen to transport fluids to and from the active tip 322.

In contrast, when the shaver side 310 is in use for a cutting operation, suction may flow via through the cutting windows to the lumen.

The inner and outer tubular members 330 and 340 are made from a sterilisable material. For example, the sterilisable material may be a metal such as stainless steel.

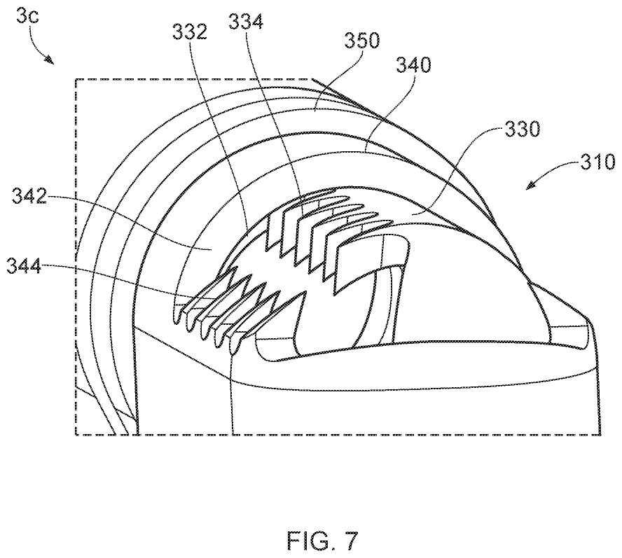

FIG. 7 shows a perspective view of the shaver side 310 of the distal end effector assembly 3c. In FIG. 7, the blade window is closing (as would be the case during shaver rotation). In FIG. 7, the inner tubular member 330 is in the process of rotating around the axis of the instrument (i.e. the central axis of the inner/outer tubular member).

FIG. 8 shows a perspective view of the shaver side 310 of the distal end effector assembly 3c. In FIG. 8, the blade window is closed (as would be the case during RF treatment). The inner cutting window 332 is not visible in FIG. 8, as it is anti-aligned with the outer cutting window 342, i.e. the inner cutting window 332 is rotated such that it is facing the opposite direction to the outer cutting window 342.

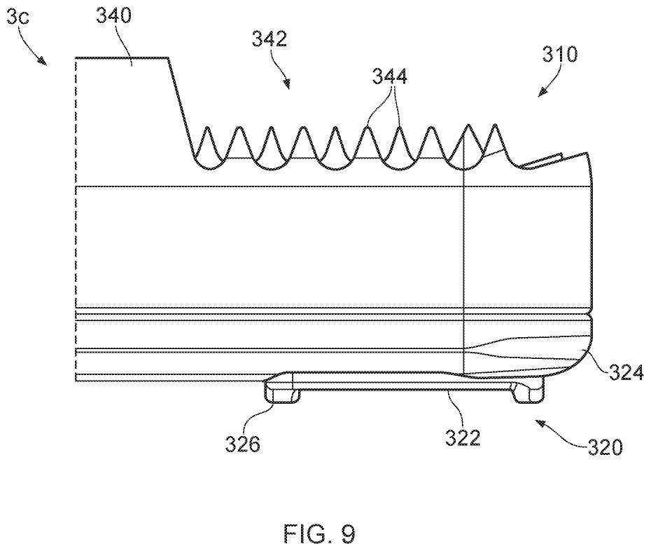

FIG. 9 shows a side view of the distal end effector assembly 3c. In FIG. 9, the blade window is open. In FIG. 9, the cutting window 342 of the outer tubular member 340 is better shown. Teeth 344 line the edge of the outer cutting window 342. The inner tubular member 330 is not visible in FIG. 9 as the blade window is open such that the inner tubular member's cutting window 332 lines up with the outer tubular member's cutting window 342. The inner tubular member 330 is therefore hidden by the outer tubular member 340 in this view.

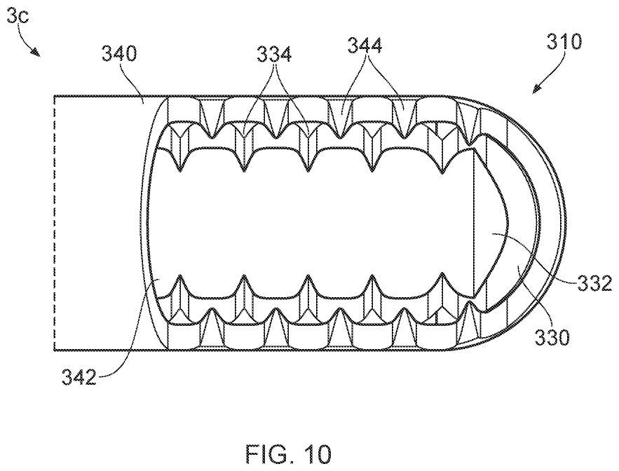

FIG. 10 shows a top view of the shaver side 310 of the distal end effector assembly 3c. In FIG. 10, the blade window is open. In FIG. 10, the cutting windows of the inner and outer tubular members (332 and 342 respectively) are aligned such that the blade window is open.

Hub Connection

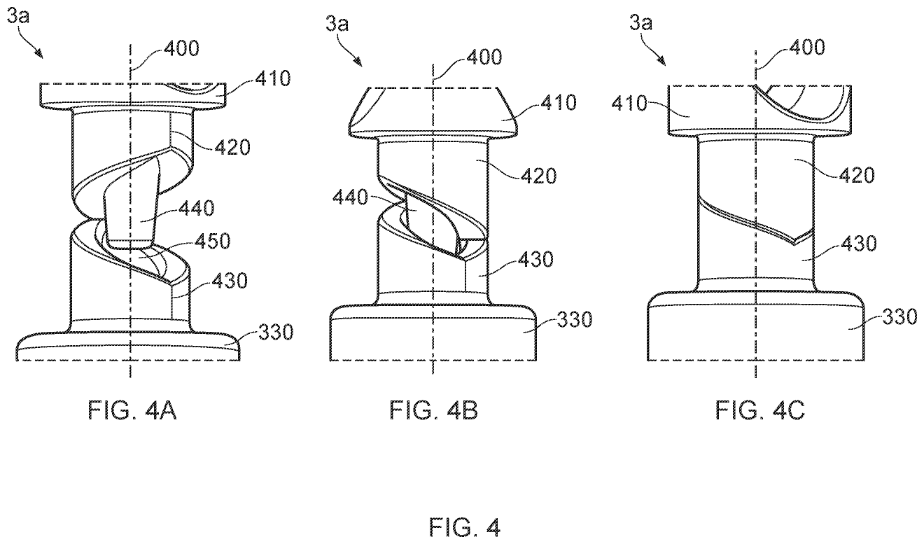

An embodiment of the invention will now be described with reference to the example surgical instrument described above. In the example surgical instrument, the RF shaver, described above, the rotary surgical end effector is the inner tubular member 330. The handpiece 3 connects to the shaft 3b of the surgical instrument via a hub connection 3a. The hub connection 3a may comprise an outer hub (not shown) which connects to the outer tubular member 340 and an inner hub 410 which connects to the inner tubular member 330 (the rotary surgical end effector in the example of the RF shaver device). The outer hub is fixed to the handpiece, whereas the inner hub 410 is loosely held in place by the handpiece such that it can be rotated by the motor drive unit. The inner hub 410 is located at the distal end of the motor drive unit. The handpiece 3 houses the motor drive unit which rotates the inner tubular member 330 by way of the drive shaft 440 which protrudes from the inner hub 410, as shown in FIG. 4. In operation, the entire inner hub 410, including the first engaging portion 420 and the drive shaft 440, is rotated by the motor. The drive shaft 440 mates with a mating portion 450 located at the proximal end of the inner tubular member 330 to transmit mechanical power, torque, and rotation to the inner tubular member 330. The drive shaft 440 and mating portion 450 are configured to share sufficient surface area to effectively communicate the motor torque to the inner tubular member 330.

The inner hub 410 has a first engaging portion 420 at its distal end which engages/couples with a second engaging portion 430 located at the proximal end of the inner tubular member 330. The drive shaft 440 protrudes from the first engaging portion 420 to mate with the mating portion 450 of the inner tubular member 330. The mating portion 450 is located within the second engaging portion 430. The engaging of the two portions 420, 430 brings the drive shaft 440 and the mating portion 450 together such that they are mated and enable torque to be transferred from the motor to the inner tubular member 330.

The configuration of the inner tubular member 330 and the inner hub 410 of the motor drive unit is such that there is only one possible rotational alignment between the inner tubular member 330 and the inner hub 410 (i.e. the motor drive unit) when the two are coupled. This is achieved using a coupling means for coupling the motor drive unit and the inner tubular member 330, the coupling means being configured such that there is only one possible rotational alignment between the inner tubular member 330 and the motor drive unit when coupled. The drive shaft 440 is fixed in relation to the inner hub 410, so this also means that there is only one possible rotational alignment between the inner tubular member 330 and the drive shaft 440.

FIG. 4 illustrates an example of such a configuration. In particular, FIG. 4 illustrates steps for coupling/engaging the inner tubular member 330 with the inner hub 410 of the motor drive unit. FIG. 4 comprises FIGS. 4A, 4B and 4C which show the engagement process step-by-step. The first and second engaging portions are complementarily shaped such that there is only one position around the rotational axis 400 where the mating portion 450 of the inner tubular member 330 and the drive shaft 440 are fully coupled/mated.

In the example of FIG. 4, the first and second engaging portions 420, 430 form a spiral arrangement wherein each of the first and second engaging portions are asymmetrical but complimentary shapes. A bidirectional spiral promotes the interconnection to rotate to the low part of the adjoining spiral, thus guaranteeing the rotational alignment. However, other geometries arranged to achieve the same function (there being only one position around the rotational axis 400 where the inner tubular member 330 and the motor drive unit are fully coupled/mated) could equally be used.

In FIG. 4A, the engaging portions are in contact but not fully engaged as the geometry of the portions 420, 430 keeps the drive shaft 440 and the mating portion 450 apart. In FIG. 4B, the inner tubular member 330 has been rotated around its rotational axis 400 relative to the inner hub 410 of the motor drive unit such that the two engaging portions 420, 430 are able to move closer together. At this stage illustrated in FIG. 4B, the drive shaft 440 and the mating portion 450 are still not fully coupled/mated, but the drive shaft 440 is inserted into the mating portion 450. In FIG. 4C, the inner tubular member 330 has been further rotated around its rotational axis 400 relative to the inner hub 410 of the motor drive unit such that the two engaging portions 420, 430 are able to fully engage. The complementary shapes of the portions 420, 430 slot together when the one possible rotational alignment between the inner tubular member 330 and the inner hub 410 is achieved. At this stage, the drive shaft 440 and the mating portion 450 are fully coupled/mated, and thus the motor drive unit and the inner tubular member 330 are connected such that mechanical power, torque, and rotation can be transmitted from the motor drive unit to the inner tubular member 330. The purpose of the engaging portions 420, 430 is to only allow mating of the drive shaft 440 and the mating portion 450 at one particular rotational alignment of the inner tubular member 330 and the inner hub 410. This means the angular/rotational position of the inner tubular member 330 is set with respect to the motor drive unit.

The motor drive unit is encoded such that the drive shaft 440 is positionable in one or more discrete rotational positions. As the inner tubular member 330 is rotationally fixed to the drive shaft 440 when coupled, this means that the inner tubular member 330 is also positionable in one or more discrete rotational positions, as the rotational positioning of the inner tubular member 330 is dictated by the rotational positioning of the drive shaft 440.

For example, as described above, when the RF side 320 is to be used as a suction tool by applying a vacuum through the lumen within the inner tubular member 330, the inner tubular member 330 (which acts as a cutting blade) is stopped from rotating and the cutting windows of the inner and outer tubular members are anti-aligned with each other, i.e. closing the cutting windows, (as is the case in FIG. 8) so that the vacuum is applied through the suction path connecting the suction aperture to the suction pump 10 via the lumen to transport fluids to and from the active tip 322. Being able to quickly and accurately position the inner tubular member 330, due to the angular alignment of the inner hub 410 and inner tubular member 330 being set, is therefore advantageous as in use this allows the surgeon to quickly switch to RF-only functionality.

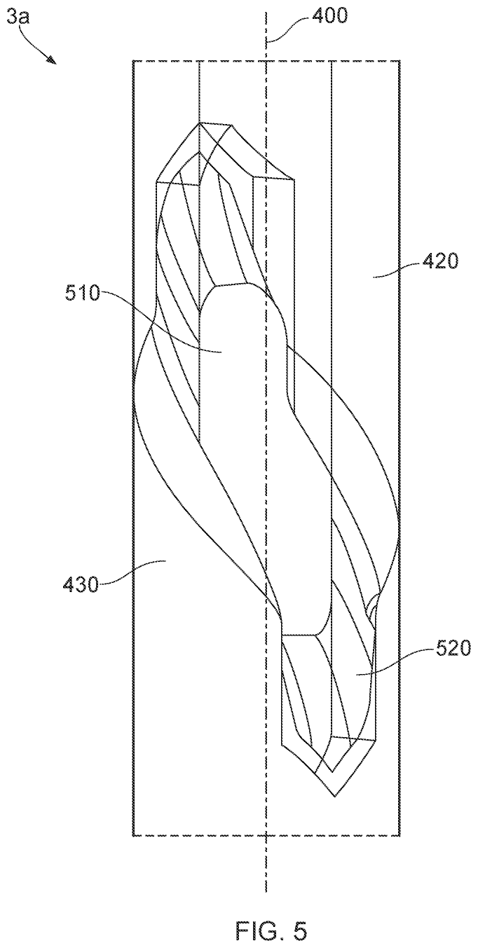

FIG. 5 illustrates how torque transfer means to increase the torque transfer capacity between the drive shaft and the inner tubular member can be added to coupling means. The increased torque transfer capacity is with respect to embodiments of the invention without the means present. Such means may comprise a protrusion 510, for example, a pin and/or a lug. The protrusion 510 may extend from the first engaging portion 420 (and/or the second engaging portion 430) to be received by a complementary-shaped receptacle 520 in the second engaging portion 430 (and/or the first engaging portion 420 respectively). When the portions 420, 430 are brought together to be engaged, the protrusion 510 initially keeps the portions 420, 430 further apart than in the embodiment illustrated in FIG. 4, until the portions 420, 430 are in their one possible rotational alignment. At this point, the protrusion 510 is received by the complementary-shaped receptacle 520, thereby slotting the portions 420, 430 together such that the portions 420, 430 are fully engaged. The torque transfer means has the advantage that the portions 420, 430 are more securely engaged, and therefore far less likely to come uncoupled.

In operation once assembled, the motor drive unit rotates the inner hub 410 which is loosely held in place by the handpiece 3. The inner hub 410, first engaging portion 420 and the drive shaft 440 are all fixed together such that when the inner hub 410 rotates, the first engaging portion 420 and the drive shaft 440 also rotate. By way of the coupling means described herein, the inner tubular member 330 is also rotatably fixed to the inner hub 410 when fully coupled, and thus the motor drive unit rotates the inner tubular member 330.

It will be apparent to the skilled person that although one advantageous use of the invention is in a dual-sided RF shaver, the invention may also be applied to instruments without RF capabilities, for example, arthroscopic shavers. Further, embodiments of the invention could equally be applied to other rotary surgical end effectors. Many surgical instruments have rotatable end effectors, e.g., surgical staplers, endoscopic cutting instruments, forceps mounted at the end of rotatable shafts, etc.

Various modifications whether by way of addition, deletion, or substitution of features may be made to above-described embodiment to provide further embodiments, any and all of which are intended to be encompassed by the appended claims.

1. A surgical instrument, comprising:
a motor drive unit;
a rotary surgical end effector arranged to be coupled to the motor drive unit; and
a coupling means for coupling the motor drive unit and the rotary surgical end effector, the coupling means being configured such that there is only one possible rotational alignment between the rotary surgical end effector and the motor drive unit when coupled.

2. The surgical instrument of claim 1, wherein the coupling means is configured such that the rotary surgical end effector is rotationally locked with the motor drive unit when coupled, such that there is a one-to-one relationship between rotation of the motor drive unit and rotation of the rotary surgical end effector.

3. The surgical instrument of claim 1, wherein the coupling means comprises a first engaging portion located at a distal end of the motor drive unit and a second engaging portion located at a proximal end of the rotary surgical end effector, the first and second engaging portions arranged to engage when coupled.

4. The surgical instrument of claim 3, wherein the motor drive unit comprises a drive shaft which protrudes from the first engaging portion, and the rotary surgical end effector comprises a mating portion configured to receive the drive shaft, the mating portion being located within the second engaging portion, the drive shaft and the mating portion being configured to mate when the rotary surgical end effector and the motor drive unit are coupled.

5. The surgical instrument of claim 3, wherein the first and second engaging portions are complementarily shaped such that there is only one position around the rotational axis where the rotary surgical end effector and the motor drive unit are fully coupled.

6. The surgical instrument of claim 5, wherein the rotary surgical end effector and the motor drive unit being fully coupled requires the drive shaft and the mating portion to mate.

7. The surgical instrument of claim 3, wherein the first and second engaging portions form a spiral arrangement.

8. The surgical instrument of claim 1, wherein the motor drive unit is encoded such that the rotary surgical end effector is positionable in one or more discrete rotational positions.

9. The surgical instrument of claim 1, wherein the rotary surgical end effector comprises a rotary shaver arrangement.

10. The surgical instrument of claim 9, wherein the rotary shaver arrangement comprises an inner tubular member arranged to mechanically cut tissue when rotated.

11. The surgical instrument of claim 10, wherein the rotary shaver arrangement comprises an outer tubular member which concentrically surrounds the inner tubular member, the outer tubular member having a central passageway and a first cutting window at the distal end thereof, the inner tubular member being rotatably mounted in the central passageway of the outer tubular member, and the inner tubular member having a second cutting window at the distal end thereof.

12. The surgical instrument of claim 8, wherein:
a) the first and second cutting windows align when the inner tubular member of the motor drive unit is positioned in a first position of the one or more discrete rotational positions, and
b) the first and second cutting windows anti-align when the inner tubular member of the motor drive unit is positioned in a second position of the one or more discrete rotational positions.

13. The surgical instrument of claim 11, wherein:
a) the first and second cutting windows align when the inner tubular member of the motor drive unit is positioned in a first position of the one or more discrete rotational positions, and
b) the first and second cutting windows anti-align when the inner tubular member of the motor drive unit is positioned in a second position of the one or more discrete rotational positions

14. The surgical instrument of claim 10, wherein a central passageway of the inner tubular member provides a central suction lumen.

15. The surgical instrument of claim 11, wherein the rotary shaver arrangement is such that, when in use, rotation of the inner tubular member within the outer tubular member causes a tissue cutting action of a cutting blade located on the first cutting window interacting with the second cutting window.

16. The surgical instrument of claim 1, wherein the surgical instrument further comprises a handpiece arranged to house the motor drive unit and wherein the handpiece is coupleable to the rotary surgical end effector via the coupling means.

17. The surgical instrument of claim 1, wherein the coupling means further comprises a torque transfer means to increase the torque transfer capacity between the motor drive unit and the rotary surgical end effector.

18. The surgical instrument of claim 17, wherein the coupling means comprises a first engaging portion located at a distal end of the motor drive unit and a second engaging portion located at a proximal end of the rotary surgical end effector, the first and second engaging portions being arranged to engage when coupled, and wherein the torque transfer means comprises a protrusion extending from one of the first and second engaging portions, arranged to be received by the other of the first and second engaging portions.

19. The surgical instrument of claim 18, wherein the protrusion comprises a pin and/or a lug.

20. An electrosurgical instrument, comprising:
a surgical instrument, comprising a motor drive unit, a rotary surgical end effector arranged to be coupled to the motor drive unit, and a coupling means for coupling the motor drive unit and the rotary surgical end effector, the coupling means being configured such that there is only one possible rotational alignment between the rotary surgical end effector and the motor drive unit when coupled;
the surgical instrument further comprising a radio frequency (RF) arrangement including an active electrode; and
an operative shaft having RF electrical connections operably connected to the active electrode.
